(19) United States
(12) Patent Application Publication
(10) Pub. No.: US 2022/0280588 A1
(43) Pub. Date: Sep. 8, 2022

(54) MEDICAMENT FOR TREATING STROKE CAUSED BY DEEP FUNGAL INFECTIONS

(71) Applicant: Mingchun XIAO, Guangdong (CN)

(72) Inventor: Mingchun XIAO, Guangdong (CN)

(21) Appl. No.: 17/632,865

(22) PCT Filed: Aug. 6, 2019
(86) PCT No.: PCT/CN2019/099352

§ 371 Date: Feb. 4, 2022

(51) Int. Cl. A61K 36/8988; A61K 36/284; A61K 36/076; A61K 36/752; A61K 36/484; A61K 36/8888; A61K 36/74; A61K 36/236; A61K 36/264; A61K 36/59; A61K 36/714; A23L 33/00; A23L 33/105; A23K 10/30

(52) U.S. Cl. CPC A61K 36/8988; A61K 36/284; A61K 36/076; A61K 36/752; A61K 36/484; A61K 36/8888; A61K 36/74; A61K 36/236; A61K 36/264; A61K 36/59; A61K 36/714; A23L 33/40; A23L 33/105; A23K 10/30; A61K 2236/37; A61K 2236/53; A23V 2002/00

(57) ABSTRACT

Use of addition and subtraction of the Banxia Baizhu Tianma Decoction in preparation of medicines or health products for treating strokes caused by deep fungal infections. The present invention relates to the field of medical and health care, and specifically relates to use of chemical ingredients, i.e. alkaloids and volatile oils contained in plant raw materials such as Gastrodia elata, Pinellia ternata, Atractylodes macrocephala, Poria cocos, fresh orange peel and Liquorice to prepare drugs or health products for treating strokes caused by deep fungal infections.

### TECHNICAL FIELD

The invention relates to the field of medical and health care, relates to new use of addition and subtraction of the “Banxia Baizhu Tianma Decoction” in preparation of medicines or health products for treating strokes caused by deep fungal infections, and specifically relates to new use of chemical ingredients, i.e. alkaloids and volatile oils contained in plant raw materials such as Gastrodia elata, Pinellia ternata, Atractylodes macrocephala, Poria cocos, fresh orange peel and Liquorice to prepare drugs or health products for treating strokes caused by deep fungal infections.

### BACKGROUND OF THE INVENTION

Modern medicine divides mycoses into two major categories: superficial mycoses and deep mycoses. Superficial mycosis refers to infections of skin keratin tissues (including stratum corneum, nail plate, hair, etc.). Deep mycosis is also called an invasive fungal disease (IFD) and refers to a fungal infectious disease caused by fungi invading human subcutaneous mucosal tissues, blood and internal organs. The most common fungal pathogens are Candida, Aspergillus and others such as Cryptococcus, Mucor and Pneumocystis, etc. According to the incomplete statistics from the Global Action Fund for Fungal Infections (GAFFI), 300 million people worldwide suffer from serious fungal infections each year, and more than 25 million people are at very high risk of death or blindness. However, researches on diseases caused by fungal infections of the brain are very limited today, and the drugs for treating deep fungal infections that are currently available on the market often have large side effects, and their long-term use cause harm to the body organs, wherein the curative effect is not ideal, while many undesirable effects appear. Furthermore, the patent “Cracking the Pathogenic Factors of Cancer and Traditional Chinese Medicine Treatment Method” to the application filed earlier by the applicant (the application No. 201310274489.7), as well as the international patent “A Pharmaceutical Composition for the Treatment of Cancer and Preparation Method thereof” (the application No. PCT/CN2013/080936, the application No. 201380075486.9 for entry into national phase in China, the patent No. U.S. Pat. No. 9,498,508B2 for entry into the United States), and the international patent “A Pharmaceutical Composition for Preventing and Treating Cancer of Reproductive System and Preparation Method thereof” (the application No. PCT/CN2013/084813, the application No. 201380053736.9 for entry into national phase in China, the patent No. U.S. Pat. No. 9,744,206B2 for entry into the United States) relate to methods of traditional Chinese medicine for treating deep fungal infections, wherein the “Banxia Baizhu Tianma Decoction” is the basic prescription for treating fungal infections, and based thereon, this application is the continuation application.

In recent years, medical researches have also proved that the main transmission routes of fungi such as Aspergillus, Cryptococcosus and Mucor are the respiratory tract and skin, which can cause systemic cryptococcosis, pulmonary aspergillosis, rhinocerebral mucormycosis, pulmonary mucormycosis, Gastrointestinal mucormycosis, disseminated mucormycosis, etc.

Modern medical researches have also proved that the main route of aspergillosis infection is to inhale spores in the air. Aspergillus spores enter the sinuses and lungs during inhalation, germinate to produce hyphae and enter cytoplasmic changes, and Aspergillus likes to invade blood vessels. “Dermatovenereology” has also confirmed that Candida albicans can cause infections of the skin, mucous membranes, internal organs, bones, and brain through exogenous or endogenous routes.

Cryptococcus neoformans is mainly inhaled through the respiratory tract. The lungs are infected first. From the lungs, it can spread to the other parts of the body, including the skin, bones, heart and so on. The most vulnerable is the central nervous system, as it leads to chronic meningitis.

Modern studies have proved that the spores of fungi multiply by germination to form hyphae, and the hyphae intertwine to form clusters. When the germinating spores grow to a certain size, they separate from the mother's body.

Apoplexy relates to cerebrovascular diseases, a group of diseases of vascular-derived brain damage, commonly known as stroke. On the 24 Jun. 2019, the world-renowned medical journal “The Lancet” published a large-scale paper on China's public health research, wherein the mortality rate of Chinese people from 1990 to 2017 and the mortality rate with respect to specific diseases are analysed. Among other things, the research identified the top ten causes of death from 282 types of deaths in 2017, and stroke became the number one cause of death in China. In December, 2018, “The New England Journal of Medicine”, the USA, published a paper describing stroke morbidity and death risks in different regions of the world, wherein the GBD data was also used.

This research was focused on first-time strokes, including ischemic, i.e. because the blood vessels leading to the brain are blocked, and hemorrhagic, due to the rupture of the fragile blood vessels. Furthermore, for the first time, the research lowered the onset age regarding the risk of stroke to 25 years, while before it was 45 years. A research published by “The Lancet” in August, 2017 showed that more than ⅓ of the adults in China suffers from high blood pressure. This research came to the same conclusion: stroke was the biggest cause of death in China, wherein ⅕ of the deaths is caused by stroke every year, and out-of-control hypertension is a big risk factor.

According to the etiology and pathology, cerebrovascular diseases can be divided into two categories, namely ischemic and hemorrhagic. The ischemic include: transient ischemic attack, infarction (thrombogenesis, cerebral embolism, lacunar infarction). The hemorrhagic include: cerebral hemorrhage, subarachnoid hemorrhage. At present, some people classify mixed strokes (simultaneous or successive hemorrhagic and ischemic lesions) as a single category. At present, the pathogenesis of stroke is not clear for the medical community. It is currently believed that the microembolism caused by microemboli that separate from atherosclerotic lesions of the walls of the large arteries in the neck may be the most common cause. In hypertensive patients, due to the poor cerebrovascular autoregulation, the cerebral vessels do not contract, and the cerebral blood flow increases significantly instead of decreasing. This hyperperfusion under the action of high blood pressure leads to increased capillary pressure and capillary destruction, which can cause severe cerebral edema and hemorrhage. The application of any vasodilator at this time is obviously harmful and useless.

The clinical manifestations of stroke show different symptoms due to different vascular lesions. The main clinical manifestations of ischemic stroke are sudden fainting, paralysis of one side of the body (hemiplegia), hemianesthesia, deviation of the eye and mouth, blindness in one eye on the same side, glossolalia, ataxia of body and limbs, blurred vision in both eyes.

Cerebral thrombosis often occurs in patients over 50 years old with cerebral arteriosclerosis, accompanied by a medical history of hypertension, diabetes and heart diseases, and occur in some patients during sleep. Severe cerebral embolism can suddenly lead to coma, high fever, intracranial pressure, edema, and cerebral hernia and death are possible.

Hypertension with arteriosclerosis, as well as arteriovenous malformation or aneurysm rupture, cerebral artery inflammatory wall necrosis and so on are most common to hemorrhagic strokes, wherein the clinical symptoms are related to the location and volume of bleeding. Headache, dizziness, limb weakness, hemiplegia, disturbance of consciousness, brain edema, coma and other clinical symptoms manifest themselves.

In traditional Chinese medicine, it is believed that the pathological mechanism of strokes is also quite complicated, and its roots lie in deficiency of the yin of the liver and kidney, zang-fu (viscera) dysfunction and imbalance of yin and yang.

The theory of yin-yang and five elements was the conception of nature in ancient China. Traditional Chinese medicine began to adopt the theory of yin and yang, approximately, at the end of the Spring and Autumn Period (the fifth century BC), and it also adopted the new theory of five elements at the late Warring States Period (the third century BC), thus forming the framework of the basic theory of Chinese medicine. According to traditional Chinese medicine, a human is an organic whole composed of yin and yang, and, in particular, can be divided into six parts: three yins and three yangs. Three yins and three yangs are respectively related to the twelve meridians of hand and foot, the five zang organs and the six fu organs. The three yin meridians of hand are: the Lung Meridian Of Hand-Taiyin, the Pericardium Meridian Of Hand-Jueyin and the Heart Meridian Of Hand-Shaoyin. The three yang meridians of hand are: the Large Intestine Meridian Of Hand-Yangming, the Sanjiao Meridian Of Hand-Shaoyang and the Small Intestine Meridian Of Hand-Taiyang. The three yang meridians of foot are: the Stomach Meridian of Foot-Yangming, the Gallbladder Meridian of Foot-Shaoyang and the Urinary Bladder Meridian of Foot-Taiyang. The three yin meridians of foot are: the Spleen Meridian of Foot-Taiyin, the Liver Meridian of Foot-Jueyin and the Kidney Meridian of Foot-Shaoyin.

According to the course direction and intersection rules of the twelve meridians and collaterals: the three yin meridians of hand run from the chest to the hand and intersect the three yang meridians of hand; the three yang meridians of hand run from the hand to the head and intersect the three yang meridians of foot; the three yang meridians of foot run from the head to the foot and intersect the three yin meridians of foot; the three yin meridians of foot run from the foot to the abdomen and chest and intersect the three yin meridians of hand. Among other things, the yin meridians and the yang meridians intersect at the ends of the limbs, the yang meridians and the yang meridians intersect at the head, and the yin meridians and the yin meridians intersect at the chest. The exterior interior relations are: the lung meridian and the large intestine meridian, the spleen meridian and the stomach meridian, the heart meridian and the small intestine meridian, the kidney meridian and the urinary bladder meridian, the pericardium meridian and the sanjiao meridian, the liver meridian and the gallbladder meridian. There are many analytical theories of the theory of yin-yang and five elements in the field of traditional Chinese medicine, but they are unable to make definite their medical principles and theoretical basis, and there has been no relevant literature to report that the yin meridians are veins and the yang meridians are arteries.

The basis of natural medicines for disease treatment and prevention lies in their active ingredients, and the chemical composition of any natural medicine is very complex. Alkaloids belong to the main active ingredients of plant medicines. There are many kinds of known alkaloids. Their structure is relatively complex. According to the biogenic relationship and chemical classification of alkaloids, alkaloids are divided into 25 categories, such as pyrrole, tropane, pyrrolizidine, piperiden, lycoprine, indolizidine, quinorizidine, acridine, amphetamine, tetrahydroisoquinoline, emetine, quinoline, peptide, terpene alkaloids, etc. Most alkaloids are alkaline, and most of alkaloids are crystalline. Alkaloids are nitrogen-containing organic compounds, most of them have a complex ring structure, and nitrogen is mostly contained in the ring. The alkaline strength is related to the structure type of alkaloids. Many scholars have found that alkaloids have many pharmacological activities, including anti-tumor, anti-virus, anti-inflammatory, antibacterial, analgesic, liver protection, cardiovascular and many other aspects. For example, morphine obtained from opium has a strong analgesic effect, codeine has an effect of relieving cough, papaverine has an effect of relaxing and smoothing muscle; ephedrine in ephedra has an antiasthmatic effect; berberine in Coptis chinensis and Phellodendron chinensis has an antibacterial effect; daturine in datura, semen hyoscyami and belladonna has an antispasmodic effect, and so on. These are the most studied alkaloids at present, and their effects are different. However, at present, the pharmacological mechanisms of anti-tumor, anti-virus, anti-inflammatory, antibacterial, analgesic, liver protection, cardiovascular effects of alkaloids have not been fully studied. Furthermore, in the related literature, it has not been reported yet that alkaloids have effects of treating deep fungal infections or strokes caused by fungal infections.

Based on the feature that alkaloid salts are soluble in water, while alkaloids are insoluble in water, methods for extracting alkaloids can be determined after extraction with organic solvents. Alkaloids are mostly soluble in organic solvents such as methanol, ethanol, diethyl ether and chloroform, but are generally insoluble or hardly soluble in water. Based on this feature, different solvents can be used to extract alkaloids from plants. The commonly used methods include cold immersion, percolation, ultrasonic extraction, Soxhlet extraction, heat reflux extraction, etc. New technologies of alkaloid extraction are: 1. Membrane separation that has the advantages of not requiring addition of chemical reagents, high separation efficiency, mild conditions, etc. 2. Supercritical fluid extraction that is characterised by high extraction efficiency, high purity, mild conditions, no pollution, and that the temperature and pressure can be changed.

Volatile oils belong to a category of ingredients that have important pharmacological effects in Chinese medicine and plants. Volatile oil is a general term for oily liquids in plants that have aromatic odour and can volatile at room temperature. They are widely distributed in the plant kingdom, especially Compositae, Rutaceae, Umbelliferae, Lamiaceae, Lauraceae, Magnoliaceae, Zingiberaceae and other plants. A volatile oil is mainly composed of terpenoids and aromatic compounds and their oxygen-containing derivatives such as alcohols, aldehydes, ketones, phenols, ethers, internal fats, etc., in addition to nitrogen-containing and sulfur-containing compounds. Modern pharmacological experiments show that: volatile oils have anti-inflammatory, anti-allergic, antimicrobial, anti-mutation, anti-cancer effects, anthelmintic effects, enzyme inhibition effects, effects on the central nervous system and effects on the respiratory system. However, the understanding and research on the activity mechanism or pharmacological mechanism of volatile oils are still very limited. Furthermore, in the related literature, it has not been reported yet that volatile oils have effects of treating deep fungal infections or strokes caused by fungal infections.

Methods for extracting volatile oils can be chosen from different extraction methods, such as: 1. Steam distillation: the extraction is performed using the volatility and water immiscibility of volatile oils. 2. Organic solvent extraction: continuous reflux extraction or cold leaching using petroleum ether, diethyl ether and other organic solvents are used for extraction. 3. Supercritical fluid extraction, wherein carbon dioxide supercritical fluid is used to extract volatile oils. 4. A cold pressing method that is suitable for extraction from fresh plants with high oil content.

The “Banxia Baizhu Tianma Decoction” is a commonly used prescription for treating wind-phlegm dizziness and headache. Using the “Banxia Baizhu Tianma Decoction” added with Acorns calamus and Curcuma longa as the basic prescription and a Chinese medicine method of adding and subtracting a decoction based on the syndrome differentiation, Sun Liting treated 40 cases of dizziness (9 cases of hypertension, 14 cases of cerebral arteriosclerosis, 11 cases of vertebrobasilar insufficiency, 3 cases of cervical spondylosis and 3 cases of Meniere's disease). The results: 19 cases were cured, 14 cases were markedly effective, 5 cases were effective and 2 cases were ineffective. [Heilongjiang Chinese medicine, 1998]. However, it has neither been reported that the “Banxia Baizhu Tianma Decoction” is used for treating strokes caused by deep fungal infections, nor has it been reported that medicines prepared by using, as the crude materials, the active pharmaceutical ingredients like mixed alkaloids and/or volatile oils contained in the raw materials of the “Banxia Baizhu Tianma Decoction” such as Pinellia ternata, Gastrodia elata, Atractylodes macrocephala, Poria cocos, orange peel and Liquorice are used for treating strokes caused by deep fungal infections, or have a thrombolytic effect.

Pinellia ternata is a drug for warming and resolving cold phlegm. The efficacy: removing dampness to reduce phlegm; calming the adverse-rising energy and preventing or arresting vomiting; dissolving lumps and resolving masses; for external use, relieving swelling and pain; treating damp phlegm and cold phlegm syndromes. According to modern researches, Pinellia ternata contains volatile oils, alkaloids and a variety of chemical components. Its alkaloids have an anti-tumor effect. Pinellia ternata alcohol extract has broad-spectrum antibacterial activity and has a certain effect regarding inhibitory action on fungi. However, there have been no reports that alkaloids and volatile oils of Pinellia ternata have an effect of treating strokes caused by deep fungal infections or have a thrombolytic effect.

Atractylodes macrocephala is a Qi-tonifying drug. Modern researches show that Atractylodes Macrocephala contains volatile oils that have a sedative effect. However, there have been no reports that alkaloids and volatile oils of Atractylodes macrocephala are used for treating strokes caused by deep fungal infections or have a thrombolytic effect.

Gastrodia elata is a drug for “relieving wind and spasm” and is a medicine for treating headache, numbness of the limbs, rheumatoid arthritis and so on. According to modern researches, volatile oils of Gastrodia elata have significant inhibitory action on Aspergillus oryzae and Aspergillus flavus. Volatile oils of Gastrodia elata have significant antibechic and good expectorant effects. However, there have been no reports that alkaloids and volatile oils of Gastrodia elata are used for treating strokes caused by deep fungal infections or have a thrombolytic effect.

Poria cocos is a drug for inducing diuresis to remove swelling. According to modern researches, Poria cocos decoctions, alcohol extracts and diethyl ether extracts have diuretic, sedative, anti-tumor and hypoglycemic effects, respectively. However, there have been no reports that alkaloids and volatile oils of Poria cocos are used for treating strokes caused by deep fungal infections or have a thrombolytic effect.

Fresh orange peel is a Qi regulating drug. According to modern researches, its volatile oil decoction has an effect of dilating the trachea, and its volatile oils have an effect of stimulating expectoration. However, there have been no reports that alkaloids and volatile oils of fresh orange peel are used for treating strokes caused by deep fungal infections or have a thrombolytic effect.

Liquorice is a Qi-tonifying drug. According to modern researches, it contains alkaloids, polysaccharides and other chemical components. However, there have been no reports that alkaloids and volatile oils of Liquorice are used for treating strokes caused by deep fungal infections or have a thrombolytic effect.

### SUMMARY OF THE INVENTION

The purpose of the present invention is to provide a medicine for treating strokes caused by deep fungal infections, specifically to provide new use of a chemical composition including alkaloids and/or volatile oils contained in plant raw materials of the “Banxia Baizhu Tianma Decoction” such as Gastrodia elata, Pinellia ternata, Atractylodes macrocephala, Poria cocos, fresh orange peel, and Liquorice to prepare drugs or health products for treating strokes caused by deep fungal infections.

Furthermore, the present invention provides a medicine for treating strokes caused by deep fungal infections, characterized in that the active pharmaceutical ingredients of the medicine include alkaloids and/or volatile oils extracted from Gastrodia elata, alkaloids and/or volatile oils extracted from Pinellia ternata, alkaloids and/or volatile oils extracted from Atractylodes macrocephala, alkaloids and/or volatile oils extracted from Poria cocos, alkaloids and/or volatile oils extracted from fresh orange peel, alkaloids and/or volatile oils extracted from Liquorice.

Method 1 for preparing the medicine is as follows: taking alkaloids of Gastrodia elata, alkaloids of Pinellia ternata, alkaloids of Atractylodes macrocephala, alkaloids of Poria cocos, alkaloids of fresh orange peel, alkaloids of Liquorice and mixing them in a certain proportion; adding various conventional excipients required to prepare different dosage forms thereto; and preparing pharmaceutically acceptable dosage forms.

Method 2 for preparing the medicine is as follows: taking volatile oils of Gastrodia elata, volatile oils of Pinellia ternata, volatile oils of Atractylodes macrocephala, volatile oils of Poria cocos, volatile oils of fresh orange peel, volatile oils of Liquorice and mixing them in a certain proportion; adding various conventional excipients required to prepare different dosage forms thereto; and preparing pharmaceutically acceptable dosage forms.

Method 3 for preparing the medicine is as follows: taking alkaloids and volatile oils of Gastrodia elata, alkaloids and volatile oils of Pinellia ternata, alkaloids and volatile oils of Atractylodes macrocephala, alkaloids and volatile oils of Poria cocos, alkaloids and volatile oils of fresh orange peel, alkaloids and volatile oils of Liquorice and mixing them in a certain proportion; adding various conventional excipients required to prepare different dosage forms thereto; and preparing pharmaceutically acceptable dosage forms.

Furthermore, in the preparation methods, methods for extracting the alkaloids can be determined after organic solvent extraction, wherein the alkaloids of plants such as Gastrodia elata, Pinellia ternata, Atractylodes macrocephala, Poria cocos, fresh orange peel and Liquorice are extracted using different solvents. Supercritical fluid extraction is the first choice, followed by membrane separation, ultrasonic extraction or Soxhlet extraction, etc.

As a method for extracting volatile oils of Gastrodia elata, Pinellia ternata, Atractylodes macrocephala, Poria cocos, and Liquorice, supercritical fluid extraction is the first choice, followed by organic solvent extraction: continuous reflux extraction or cold leaching using petroleum ether, diethyl ether, etc. organic solvents, or steam distillation is chosen; and regarding fresh orange peel, cold pressing method is the first choice.

Furthermore, the medicine provided by the invention is characterized in that the weight parts of the alkaloids in the active pharmaceutical ingredients of the medicine are: 0.01-50 weight parts of alkaloids of Gastrodia elata, 0.01-50 weight parts of alkaloids of Pinellia ternata, 0.01-50 weight parts of alkaloids of Atractylodes macrocephala, 0.01-50 weight parts of alkaloids of Poria cocos, 0.01-50 weight parts of alkaloids of fresh orange peel, 0.01-50 weight parts of alkaloids of Liquorice.

Furthermore, the medicine provided by the invention is characterized in that the weight parts of the volatile oils in the active pharmaceutical ingredients of the medicine are: 0.01-50 weight parts of volatile oils of Gastrodia elata, 0.01-50 weight parts of volatile oils of Pinellia ternata, 0.01-50 weight parts of volatile oils of Atractylodes macrocephala, 0.01-50 weight parts of volatile oils of Poria cocos, 0.01-50 weight parts of volatile oils of fresh orange peel, 0.01-50 weight parts of volatile oil of Liquorice.

Furthermore, the medicine provided by the invention is characterized in that a dose of the medicine to be taken daily is further preferable as: the weight of alkaloids and volatile oils contained in 50 g of Gastrodia elata, the weight of alkaloids and volatile oils contained in 35 g of Pinellia ternata, the weight of alkaloids and volatile oils contained in 30 g of Atractylodes macrocephala, the weight of alkaloids and volatile oils contained in 30 g of Poria cocos, the weight of alkaloids and volatile oils contained in 40 g of fresh orange peel, the weight of alkaloids and volatile oils contained in 25 g of Liquorice.

A specific preparation method: from an amount that is enough to be used for 10 days, 500 g of Gastrodia elata, 350 g of Pinellia ternata, 300 g of Atractylodes macrocephala, 300 g of Poria cocos, 400 g of fresh orange peel and 250 g of Liquorice are taken to extract their respective alkaloids and volatile oils, then the extracted alkaloids are mixed and added with various conventional excipients required to prepare different dosage forms, and pharmaceutically acceptable dosage forms are prepared; the extracted volatile oils are mixed and added with various conventional excipients required to prepare different dosage forms, and pharmaceutically acceptable dosage forms are prepared.

Furthermore, the medicine is characterized in that, according to clinical symptoms, the medicine can be selectively added with alkaloids and/or volatile oils contained in raw materials such as Uncaria rhynchophylla, Ligusticum wallichii, monkshood, Berba aristolochiae mollissimae and Caulis sinomenii.

Furthermore, the medicine is characterized in that the method for preparing the medicine is as follows: extracting alkaloids and/or volatile oils from various plant raw materials such as Gastrodia elata, Pinellia ternata, Atractylodes macrocephala, Poria cocos, fresh orange peel and Liquorice to prepare the medicine, and preparing pharmaceutically acceptable dosage forms such as tablets, powder injections, soft capsules, granules or capsules using conventional methods for preparing pharmaceutical chemicals.

Furthermore, the medicine is characterized in that the method for preparing the medicine is as follows: extracting alkaloids and/or volatile oils from various plant raw materials such as Gastrodia elata, Pinellia ternata, Atractylodes macrocephala, Poria cocos, fresh orange peel and Liquorice or Uncaria rhynchophylla or Ligusticum wallichii or monkshood or Berba aristolochiae mollissimae or Caulis sinomenii to prepare the medicine, and preparing pharmaceutically acceptable dosage forms such as tablets, powder injections, soft capsules, granules or capsules using conventional methods for preparing pharmaceutical chemicals.

The scope of application of the medicines or health products for treating strokes caused by deep fungal infections includes applications in medical drugs, health foods, health care products, animal drugs, and animal feed.

The pharmacology of the raw materials of the medicine provided by the invention is as follows:

The creative idea of the invention starts from the research of “Headache Case” by Zhang Yuansu, a famous doctor of Jin Dynasty, thus explaining the “Theory of yin-yang and five elements” of ancient Chinese traditional culture. For the details of the explained content of the “Theory of yin-yang and five elements” please refer to the Application 201410299118.9 “A Broad Spectrum Anti-Anaerobic Drug Composition” filed by the applicant, which introduces the relationship between the “Theory of yin-yang and five elements” and modern medicine in detail. The “Theory of yin-yang and five elements” is the basis of the ancient Chinese medicine theory. Because the ancients called the pathogenic bacteria living in yin meridians “yin pathogens”, the “yin pathogens” were divided into “damp pathogens”, “cold pathogens”, “wind pathogens”, etc. Inspired by the explained “yin-yang theory”, the applicant believes that in traditional Chinese medicine, the “yin meridians” are veins and the “yang meridians” are arteries. The “damp pathogens” in the yin meridians refer to all kinds of pathogenic bacteria that live in the veins and are resistant to oxygen lack, such as fungi, anaerobic bacteria, viruses, bacilli, etc.; the “cold pathogens” refer to fungi (moulds); the “wind pathogens” refer to pathogenic bacteria that can be transmitted through air. Therefore, the applicant believes that “common cold due to wind-cold” in traditional Chinese medicine is caused by fungi such as Aspergillus, Candida, Cryptococcus, etc., that infect the upper respiratory tract. There are abundant blood vessels in the brain. According to the course direction and intersection rules of the theory of the twelve meridians in traditional Chinese medicine, the yang meridians cross each other at the head, that is, the arteries cross each other at the head. Therefore, it is very easy for fungi to enter the cerebral blood vessels, wherein the fungi have a strong destructive effect and damage the vascular walls, which makes the blood vessels lose their elasticity, leading to atherosclerosis or rupture thereof followed by bleeding.

Modern studies have proved that the spores of fungi multiply by germination to form hyphae, and the hyphae intertwine to form clusters. When the germinating spores grow to a certain size, they separate from the mother's body. Therefore, the applicant believes that detached fungal spore clusters are equivalent to emboli that form in the blood and are able to freely flow therein, that is, “microemboli that separate from atherosclerotic lesions of the walls of the large arteries in the neck cause microembolization of the small blood vessels and block the blood circulation”. Furthermore, atherosclerosis of the walls of the large arteries is mainly a chronic inflammatory reaction caused by fungi. The fungi multiply on a damaged artery wall, and the separated hyphal clusters form microemboli that block the blood circulation at the venules or arterioles. The reason why cerebral hemorrhage and cerebral apoplexy easily occur at night is that the fungi that stay in the head multiply in large numbers at night because the patient's body temperature is relatively low and the blood flow rate is slow, which leads to accelerated corrosion of the cerebral blood vessels and formation of more hyphal clusters that obstruct the blood circulation in the head causing an autoimmune reaction in the body that results in high blood pressure. The effect of the high blood pressure is equivalent to that of a water pump. It can make the blood flow up to the brain. Furthermore, the intracranial hypertension makes the emboli formed by the fungi to block or rupture the blood vessels that leads to a stroke. Therefore, the applicant believes that the main pathogenic factors of hypertension and strokes are caused by fungal infections of the blood vessels in the brain. Therefore, the stroke treatment principle lies in treating deep fungal infections.

Based on the characteristics of moulds that they like acidic and alkalescent conditions, and that pH 4-6 is most suitable for fungi, which means fungi do not adapt to survive in alkaline environment, it is concluded that alkaloids contained in plant raw materials are the main antifungal chemical components; and based on “removing dampness by means of aromatics, removing dampness by means of pungent, removing coldness by means of warm” of traditional Chinese medicine, it is concluded that volatile oil contained in plant raw materials are also antifungal chemical components.

Gastrodia elata is a drug for relieving wind and spasm, and it belongs to the liver meridian. Because fungi can cause disseminated blood infection and there is no oxygen in the veins because of slow blood flow, fungi are more likely to stay in the veins and multiply therein, and the separated spore clusters are more likely to block the venules. The applicant believes that the liver meridian comes out of the portal venous system of the liver, so the liver meridian is a vein that is the most vulnerable to fungal infection. Ancient Chinese doctors defined the circulation path of the liver meridian as follows: it starts from the big toe of the foot, ascends to the hepatic veins through the anterior medial aspect of the lower leg, then ascends along the throat into the nasopharynx, connects to the eye system and meets the Du meridian at the top of the head; and a branch of the liver meridian ascends from the liver and connects to the pulmonary veins. Therefore, the liver meridian is an important group of veins ascending from the foot to the top of the head. The lower limb segment of the liver meridian is similar to the small saphenous vein in modern medicine. Because of the abundant blood vessels in the brain, the brain is also a place where fungi are easy to stay, and the blood circulation of the eyes is equivalent to the microcirculation at the end of the brain, and the acupoints at the end of the liver meridian are in the eyes, so pathological changes of the liver meridian are the first to occur in the eyes, and such symptoms as increased intraocular pressure, cataract, blindness and so on are found. The main pathological change of stroke is cerebral vessel damage. Due to the blood-brain barrier, many medicines can not enter the brain. Gastrodia elata can pass the blood-brain barrier and is an antifungal medicine for targeted treatment of the brain. Therefore, the alkaloids and volatile oils of Gastrodia elata belong to those that can treat the fungal infection of the group of the veins of the liver meridian, especially treat fungal infection of the blood vessels in the brain and the upper respiratory tract, can kill fungi and have a thrombolytic effect. In the medicine provided by the invention, the alkaloids and volatile oils of Gastrodia elata are the sovereign drugs.

Pinellia ternata is a drug for warming and resolving cold phlegm. The drug is pungent and warm, and it belongs to the spleen, stomach and lung meridians. It is mainly used to treat the syndromes of damp phlegm and cold phlegm. Because fungi can cause a disseminated infection of the whole body through the blood circulation, the spleen meridian and the lung meridian are important veins. The stomach meridian is an artery accompanying the spleen meridian, so the spleen, stomach and lung meridians are also easily susceptible to fungal infections. The applicant believes that the spleen meridian comes out of the portal venous system of the spleen. Ancient Chinese doctors defined the circulation path of the spleen meridian as follows: it starts from the medial end of the big toe, runs along the anterior medial aspect of the thigh and enters the abdominal cavity and the spleen, then ascends to the tongue; and its branch runs from the stomach to the chest and connects to the cardiac veins. The vein of the lower limb segment of the spleen meridian is similar to the great saphenous vein in modern medicine. The circulation path of the stomach meridian is as follows: it starts from the lateral side of the wing of the nose, runs upwards and connects to the urinary bladder meridian; from the pupil, it runs straight down to the mouth, then in front of the ear to the forehead; its branch goes down from the Daying point to the Dazhui; from the supraclavicular fossa, it runs deeply into the body cavity to the stomach and the spleen; then it runs down to the groin, extends along the front of the lower limb down to the outer end of the middle toe, and connects to the spleen meridian. The lower limb segment of the stomach meridian is similar to the anterior tibial artery in modern medicine. The circulation path of the lung meridian is as follows: it starts from the Zhongfu, ascends to the throat, runs from the upper part of the chest to the front of the armpit and along the anterior medial aspect of the upper arm, and terminates at the radial end of the thumb. The lung meridian comes out of the venous system of the lungs, and the upper limb segment of the lung meridian is similar to the cephalic vein in modern medicine. A clinical symptom of a stroke is deviation of the eye and mouth that correspond to the tongue end portion of the spleen meridian and the throat portion of the lung meridian. In the case of such a clinical symptom of a stroke as hemiplegia, the numbness of the limbs is caused by blocking of the blood circulation of the upper and lower limbs. Regarding to the blood circulation at the hand and foot ends of the twelve meridians, there are very abundant capillary blood vessels. There are many venules and arterioles, and, in particular, the blood circulation of lower limbs can easily make emboli formed by loose fungal spore clusters block the microcirculation. Varicose veins of lower limbs are caused by the same reason. The acupoints on the twelve meridians are places where fungal spore clusters are easily detained and multiply with blood clots being formed. It is believed that loose fungal spore clusters usually block the venules first and then reach the arterioles. Because there are many substitutions of collateral circulation in the microcirculation, the clinical symptoms are not obvious. However, if the main veins or the main arteries are blocked by the clusters, there will be obvious clinical symptoms. For example, if the blood circulation move clusters to the heart and they block a main artery or a main vein of the heart, a myocardial infarction will occur; if they reach the lungs and block a main vein or a main artery of the lungs, a pulmonary infarction will occur; if they reach the brain and block a main artery of the brain, a cerebral infarction will occur. In particular, if a main artery is blocked, there will be a life risk. Therefore, alkaloids and volatile oils of Pinellia ternata can be used in targeted treatment of fungal infections in the blood of the lungs, spleen and stomach, can kill fungi, and have a thrombolytic effect. A team synergistic effect is provided when Pinellia ternata is used together with Gastrodia elata in treatment of fungal infections in the liver meridian. In the medicine provided by the invention, alkaloids and volatile oils of Pinellia ternata together with alkaloids and volatile oils of Gastrodia elata provide the sovereign drug.

Atractylodes macrocephala is a Qi-tonifying drug, and it belongs to the spleen and stomach meridians. In traditional Chinese medicine, it is believed that “spleen deficiency leads to phlegm-dampness”, in other words, fungi invade the blood vessels of the liver and cause a disseminated blood infection. After the infection of the lungs, pulmonary inflammatory reaction takes place, and the irritation produces a large amount of sputum that blocks the respiratory tract, making the exchange of oxygen and carbon dioxide difficult. “Qi deficiency” is oxygen lack, and the use of a Qi tonifying drug Atractylodes macrocephala together with a Qi regulating drug “fresh orange peel” can promote the exchange of oxygen and carbon dioxide in the blood. Fresh orange peel belongs to the spleen meridian and the lung meridian. Therefore, alkaloids and volatile oils of Atractylodes macrocephala and fresh orange peel are ministerial drugs in the technical solution provided by the invention, assist Pinellia ternata to restore the blood circulation of the lung, spleen and stomach meridians, and have a thrombolytic effect.

Poria cocos is a drug for inducing diuresis to remove swelling, and belongs to the heart, spleen and kidney meridians, and it is a necessary drug for strengthening the spleen and removing dampness. The heart meridian is a vein; it starts from the heart and ends at the outside end of the ring finger. That is, the heart meridian comes out of what is similar to the orifice of the superior vena cava in modern medicine, and its upper limb segment is similar to the vena basilica. The kidney meridian comes out of the renal portal venous system. Kidney meridian starts from the little toe of the foot, enters the lungs, enters the chest, and connects to the heart meridian. Its lower limb segment vein is similar to the deep vein of the foot in modern medicine. Since fungi infect the brain and cause inflammatory edema, wherein a large amount of liquid accumulates in the brain, which leads to brain edema, while Poria cocos induces diuresis to reduce edema, alkaloids and volatile oils of Poria cocos are drugs for targeted treatment of fungal infections in the heart meridian, spleen meridian and kidney meridian; they assist Gastrodia elata, Pinellia ternata and Atractylodes macrocephala to enhance the exchange of oxygen and carbon dioxide and to restore the blood circulation of the heart, spleen and kidney meridian, and have a thrombolytic effect.

Liquorice and Atractylodes macrocephalas both are Qi-tonifying drugs; Liquorice belongs to the heart, lung, spleen and stomach meridians. It has the effect of relieving cough and removing phlegm, and it also has the effect of harmonizing drug properties. Alkaloids and essential oils of Liquorice assist Atractylodes macrocephala to enhance the exchange of oxygen and carbon dioxide in the blood, can restore the blood circulation of the heart, lung, spleen and stomach meridians, and have a thrombolytic effect.

Uncaria rhynchophylla and Gastrodia elata are both drugs for calming wind and relieving spasm, and they belong to the liver and pericardium meridians. The pericardium meridian starts from the chest, ends at the end of the middle finger and is equivalent to the median cubital vein. Therefore, alkaloids and volatile oils of Uncaria rhynchophylla can assist Gastrodia elata to restore the blood circulation and reduce the blood pressure, and have a thrombolytic effect. Modern medicine has proved that its alkaloids have the effect of reducing pressure, but there is no related reports that its alkaloids have an antifungal effect.

Ligusticum wallichii is a drug for activating blood circulation and removing blood stasis, and it belongs to the liver, gallbladder and pericardium meridians. The gallbladder meridian is an artery. The gallbladder meridian starts from the eye and ends at the big toe of the foot. It is an artery accompanying the liver meridian. Therefore, alkaloids and volatile oils of Ligusticum wallichii can restore the blood circulation of the liver, gallbladder and pericardium meridians, and have a thrombolytic effect. The medicine provided by the invention can be selectively used to assist Gastrodia elata to restore the blood circulation.

Monkshood is a drug for dispelling wind, cold and dampness, and it belongs to the heart, liver, kidney and spleen meridians. It contains a variety of alkaloids and has obvious anti-inflammatory and analgesic effects. When severe stroke symptoms are exhibited, the drug can be used selectively. Its alkalinity is relatively strong, and a too high dosage thereof causes a toxic reaction easily. Alkaloids and volatile oils of monkshood can assist Gastrodia elata and Pinellia ternata to restore the blood circulation of the heart, liver, kidney and spleen meridians, can kill fungi, and have a thrombolytic effect.

Berba aristolochiae mollissimae is also a drug for dispelling wind, cold and dampness, and it belongs to the liver meridian. The alkaloids contained in Berba aristolochiae mollissimae have an obvious anti-swelling effect on arthritis in rats. In the medicine provided by the invention, when varicose veins or embolism of lower limbs cause edema of lower limbs, an amount of Berba aristolochiae mollissimae can be selectively added. Alkaloids and volatile oils of Berba aristolochiae mollissimae can assist Gastrodia elata to restore the blood circulation of the lower limb segment of the liver meridian, and have a thrombolytic effect.

Caulis sinomenii is also a drug for dispelling wind, cold and dampness. It belongs to the liver and spleen meridians and has a relatively strong effect of dredging meridians. Alkaloids of Caulis sinomenii has anti-inflammatory, analgesic, sedative, and antibechic effects. It has obvious and antagonistic effects on arrhythmia. The antihypertensive effect of total alkaloids of Caulis sinomenii is rapid and strong. However, the anti-inflammatory mechanism of Caulis sinomenii is not completely clear in the existing technology. In the medicine provided by the invention, when varicose veins or embolism of lower limbs cause edema of lower limbs, alkaloids and volatile oils of Berba aristolochiae mollissimae and Caulis sinomenii can be selectively added to assist alkaloids and volatile oils of Gastrodia elata and Pinellia ternata to restore the blood circulation of the lower limb segment of the liver and spleen meridians, have a thrombolytic effect, and eliminate inflammatory edema caused by fungi.

### SPECIFIC IMPLEMENTATION MODE

### Embodiment 1

The active pharmaceutical ingredients of the medicine for treating strokes caused by deep fungal infections in the embodiment include: alkaloids contained in 50 g of Gastrodia elata, alkaloids contained in 35 g of Pinellia ternata, alkaloids contained in 30 g of Atractylodes macrocephala, alkaloids contained in 30 g of Poria cocos, alkaloids contained in 40 g of fresh orange peel, and alkaloids contained in 25 g of Liquorice.

According to the weight parts of the selected raw materials, alkaloids were extracted from 50 g of Gastrodia elata, 35 g of Pinellia ternata, 30 g of Atractylodes macrocephala, 30 g of Poria cocos, 40 g of fresh orange peel, and 25 g of Liquorice respectively using supercritical fluid extraction or membrane separation. The alkaloids were mixed and added with conventional excipients, and tablets were prepared according to a conventional process for preparing pharmaceutical chemicals.

### Embodiment 2

The active pharmaceutical ingredients of the medicine for treating strokes caused by deep fungal infections in the embodiment include: volatile oils contained in 50 g of Gastrodia elata, volatile oils contained in 35 g of Pinellia ternata, volatile oils contained in 30 g of Atractylodes macrocephala, volatile oils contained in 30 g of Poria cocos, volatile oils contained in 40 g of fresh orange peel, and volatile oils contained in 25 g of Liquorice.

According to the weight parts of the selected raw materials, volatile oils were extracted from 50 g of Gastrodia elata, 35 g of Pinellia ternata, 30 g of Atractylodes macrocephala, 30 g of Poria cocos, and 25 g of Liquorice respectively using supercritical fluid extraction or organic solvent extraction. Volatile oils were extracted from 40 g of fresh orange peel using a cold pressing method. Then the volatile oils were mixed and added with conventional excipients, and soft capsules or granules were prepared according to a conventional process for preparing pharmaceutical chemicals.

### Embodiment 3

The active pharmaceutical ingredients of the medicine for treating strokes caused by deep fungal infections in the embodiment include: alkaloids contained in 30 g of Gastrodia elata, alkaloids contained in 30 g of Pinellia ternata, alkaloids contained in 20 g of Atractylodes macrocephala, alkaloids contained in 20 g of Poria cocos, alkaloids contained in 30 g of fresh orange peel, alkaloids contained in 15 g of Liquorice, alkaloids contained in 20 g of Uncaria rhynchophylla, and alkaloids contained in 15 g of Ligusticum wallichii.

According to the weight parts of the selected raw materials, alkaloids were extracted from 30 g of Gastrodia elata, 30 g of Pinellia ternata, 20 g of Atractylodes macrocephala, 20 g of Poria cocos, 30 g of fresh orange peel, 15 g of Liquorice, 20 g of Uncaria rhynchophylla, and 15 g of Ligusticum wallichii respectively using supercritical fluid extraction or membrane separation. The alkaloids were mixed and added with conventional excipients, and tablets were prepared according to a conventional process for preparing pharmaceutical chemicals.

### Embodiment 4

The active pharmaceutical ingredients of the medicine for treating strokes caused by deep fungal infections in the embodiment include: volatile oils contained in 30 g of Gastrodia elata, volatile oils contained in 30 g of Pinellia ternata, volatile oils contained in 20 g of Atractylodes macrocephala, volatile oils contained in 20 g of Poria cocos, volatile oils contained in 30 g of fresh orange peel, and volatile oils contained in 15 g of Liquorice, volatile oils in 20 g of Uncaria rhynchophylla, and volatile oils contained in 15 g of Ligusticum wallichii.

According to the weight parts of the selected raw materials, volatile oils were extracted from 30 g of Gastrodia elata, 30 g of Pinellia ternata, 20 g of Atractylodes macrocephala, 20 g of Poria cocos, 15 g of Liquorice, 20 g of Uncaria rhynchophylla, and 15 g of Ligusticum wallichi respectively using supercritical fluid extraction or organic solvent extraction. Volatile oils were extracted from 30 g of fresh orange peel using a cold pressing method. Then the volatile oils were mixed and added with conventional excipients, and soft capsules or granules were prepared according to a conventional process for preparing pharmaceutical chemicals. Embodiment 5

The active pharmaceutical ingredients of the medicine for treating strokes caused by deep fungal infections in the embodiment include: alkaloids contained in 25 g of Gastrodia elata, alkaloids contained in 15 g of Pinellia ternata, alkaloids contained in 15 g of Atractylodes macrocephala, alkaloids contained in 15 g of Poria cocos, alkaloids contained in 20 g of fresh orange peel, alkaloids contained in 10 g of Liquorice, alkaloids contained in 20 g of Ligusticum wallichii, and alkaloids contained in 10 g of monkshood.

According to the weight parts of the selected raw materials, alkaloids were extracted from 25 g of Gastrodia elata, 15 g of Pinellia ternata, 15 g of Atractylodes macrocephala, 15 g of Poria cocos, 20 g of fresh orange peel, 10 g of Liquorice, 20 g of Ligusticum wallichii, and 10 g of monkshood respectively using supercritical fluid extraction or membrane separation. The alkaloids were mixed and added with conventional excipients, and tablets were prepared according to a conventional process for preparing pharmaceutical chemicals.

### Embodiment 6

The active pharmaceutical ingredients of the medicine for treating strokes caused by deep fungal infections in the embodiment include: volatile oils contained in 25 g of Gastrodia elata, volatile oils contained in 15 g of Pinellia ternata, volatile oils contained in 15 g of Atractylodes macrocephala, volatile oils contained in 15 g of Poria cocos, volatile oils contained in 25 g of fresh orange peel, volatile oils contained in 10 g of Liquorice, volatile oils contained in 20 g of Ligusticum wallichii, and volatile oils contained in 10 g of monkshood.

According to the weight parts of the selected raw materials, volatile oils were extracted from 25 g of Gastrodia elata, 15 g of Pinellia ternata, 15 g of Atractylodes macrocephala, 15 g of Poria cocos, 10 g of Liquorice, 20 g of Ligusticum wallichii, and 10 g of monkshood respectively using supercritical fluid extraction or organic solvent extraction. Volatile oils were extracted from 25 g of fresh orange peel using a cold pressing method. Then the volatile oils were mixed and added with conventional excipients, and soft capsules or granules were prepared according to a conventional process for preparing pharmaceutical chemicals.

### Embodiment 7

The active pharmaceutical ingredients of the medicine for treating strokes caused by deep fungal infections in the embodiment include: alkaloids contained in 20 g of Gastrodia elata, alkaloids contained in 15 g of Pinellia ternata, alkaloids contained in 15 g of Atractylodes macrocephala, alkaloids contained in 10 g of Poria cocos, alkaloids contained in 20 g of fresh orange peel, alkaloids contained in 10 g of Liquorice, alkaloids contained in 15 g of Uncaria rhynchophylla, alkaloids contained in 15 g of Ligusticum wallichii, alkaloids contained in 10 g of monkshood, alkaloids contained in 15 g of Berba aristolochiae mollissimae, and alkaloids contained in 15 g of Caulis sinomenii.

According to the weight parts of the selected raw materials, alkaloids were extracted from 20 g of Gastrodia elata, 15 g of Pinellia ternata, 15 g of Atractylodes macrocephala, 15 g of Poria cocos, 20 g of fresh orange peel, 10 g of Liquorice, 15 g of Uncaria rhynchophylla, 15 g of Ligusticum wallichii, 10 g of monkshood, 15 g of Berba aristolochiae mollissimae, and 15 g of Caulis sinomenii respectively using supercritical fluid extraction or membrane separation. The alkaloids were mixed and added with conventional excipients, and tablets were prepared according to a conventional process for preparing pharmaceutical chemicals.

### Embodiment 8

The active pharmaceutical ingredients of the medicine for treating strokes caused by deep fungal infections in the embodiment include: volatile oils contained in 20 g of Gastrodia elata, volatile oils contained in 15 g of Pinellia ternata, volatile oils contained in 15 g of Atractylodes macrocephala, volatile oils contained in 15 g of Poria cocos, volatile oils contained in 20 g of fresh orange peel, volatile oils contained in 10 g of Liquorice, volatile oils contained in 15 g of Uncaria rhynchophylla, volatile oils contained in 15 g of Ligusticum wallichii, volatile oils contained in 10 g of monkshood, volatile oils contained in 15 g of Berba aristolochiae mollissimae, and volatile oils contained in 15 g of Caulis sinomenii.

According to the weight parts of the selected raw materials, volatile oils were extracted from 20 g of Gastrodia elata, 15 g of Pinellia ternata, 15 g of Atractylodes macrocephala, 15 g of Poria cocos, 10 g of Liquorice, 15 g of Uncaria rhynchophylla, 15 g of Ligusticum wallichii, 10 g of monkshood, and 15 g of Berba aristolochiae mollissimae respectively using supercritical fluid extraction or organic solvent extraction. Volatile oils were extracted from 20 g of fresh orange peel and 15 g Caulis sinomenii using a cold pressing method. Then the volatile oils were mixed and added with conventional excipients, and soft capsules or granules were prepared according to a conventional process for preparing pharmaceutical chemicals.

### Embodiment 9

The active pharmaceutical ingredients of the medicine for treating strokes caused by deep fungal infections in the embodiment include: alkaloids contained in 15 g of Gastrodia elata, alkaloids contained in 15 g of Pinellia ternata, alkaloids contained in 10 g of Atractylodes macrocephala, alkaloids contained in 10 g of Poria cocos, alkaloids contained in 15 g of fresh orange peel, alkaloids contained in 10 g of Liquorice, alkaloids contained in 12 g of Ligusticum wallichii, alkaloids contained in 12 g of Berba aristolochiae mollissimae, and alkaloids contained in 15 g of Caulis sinomenii.

According to the weight parts of the selected raw materials, alkaloids were extracted from 15 g of Gastrodia elata, 15 g of Pinellia ternata, 10 g of Atractylodes macrocephala, 10 g of Poria cocos, 15 g of fresh orange peel, 10 g of Liquorice, 12 g of Ligusticum wallichii, 12 g of Berba aristolochiae mollissimae, and 15 g of Caulis sinomenii respectively using supercritical fluid extraction or membrane separation. The alkaloids were mixed and added with conventional excipients, and tablets were prepared according to a conventional process for preparing pharmaceutical chemicals.

### Embodiment 10

The active pharmaceutical ingredients of the medicine for treating strokes caused by deep fungal infections in the embodiment include: volatile oils contained in 15 g of Gastrodia elata, volatile oils contained in 15 g of Pinellia ternata, volatile oils contained in 10 g of Atractylodes macrocephala, volatile oils contained in 10 g of Poria cocos, volatile oils contained in 15 g of fresh orange peel, volatile oils contained in 10 g of Liquorice, volatile oils contained in 12 g of Ligusticum wallichii, volatile oils contained in 12 g of Berba aristolochiae mollissimae, and volatile oils contained in 15 g of Caulis sinomenii.

According to the weight parts of the selected raw materials, volatile oils were extracted from 15 g of Gastrodia elata, 15 g of Pinellia ternata, 10 g of Atractylodes macrocephala, 10 g of Poria cocos, 10 g of Liquorice, 12 g of Ligusticum wallichii, 12 g of Berba aristolochiae mollissimae respectively using supercritical fluid extraction or organic solvent extraction. Volatile oils were extracted from 15 g of fresh orange peel and 15 g Caulis sinomenii using a cold pressing method. Then the volatile oils were mixed and added with conventional excipients, and soft capsules or granules were prepared according to a conventional process for preparing pharmaceutical chemicals.

Of course, the embodiments above are only for the purpose of clearly illustrating the examples given, rather than limiting the implementation mode. Based upon the above description, other different changes or modifications can be also made by those skilled in the art. It is unnecessary and impossible to present all possible embodiments herein, and the obvious changes or modifications thus extended are still within the scope of protection of the present invention.

Inventive Step and Novelty of the Invention

The inventive step and novelty of the medicines provided by the present invention lie in the following: the medicines provided by the present invention are prepared by extracting alkaloids and/or volatile oils from plant raw materials such as Gastrodia elata, Pinellia ternata, Atractylodes macrocephala, Poria cocos, fresh orange peel, and Liquorice to prepare pharmaceutical chemicals, that is, alkaloids of Gastrodia elata, alkaloids of Pinellia ternata, alkaloids of Atractylodes macrocephala, alkaloids of Poria cocos, alkaloids of fresh orange peel, alkaloids of Liquorice are mixed in a certain proportion and are added with conventional excipients, and tablets are prepared according to a conventional process for preparing pharmaceutical chemicals. Compared with the existing pharmaceutical chemicals, which are medicines made from alkaloids extracted from a single plant, it is a significant improvement. It can reduce the content of a single alkaloid in a medicine and reduce the toxic reaction caused by the excessive dosage of the single alkaloid. For example, morphine obtained from opium has a strong analgesic effect, because it is a single alkaloid, and its dose has to be too large in order to achieve the curative effect, and the toxic side effects appear easily. Moreover, the medicines provided by the invention can have a more effective targeting action on different organs and vascular tissues because of the synergistic effect of the multiple alkaloids.

Compared with traditional Chinese medicines in form of tablets, capsules, granules and the like prepared using modern methods for preparing drugs, the medicines provided by the present invention show significant progress. Not all Chinese medicines are necessarily obtained from herbs, but also from animals and minerals. The main feature of modern Chinese medicines is that it is possible to know only the main ingredients, that is, to know the chemical ingredients that can cure diseases rather than to know all the ingredients. The chemical ingredients of the medicines provided by the present invention are only alkaloids and volatile oils.

The pharmacological mechanism of the medicines provided by the invention is clear. The pharmacological mechanism of alkaloids and volatile oils contained in Gastrodia elata, Pinellia ternata, Atractylodes macrocephala, Poria cocos, fresh orange peel, Liquorice, Uncaria rhynchophylla, Ligusticum wallichii, monkshood, Berba aristolochiae mollissimae and Caulis sinomenii is that of antifungal drugs. The inventor thinks that strokes are caused by deep fungal infections, and emboli in the blood are hyphal clusters that are formed by fungal multiplication and fall into the blood. Furthermore, alkaloids and volatile oils contained in Gastrodia elata, Pinellia ternata, Atractylodes macrocephala, Poria cocos, fresh orange peel, Liquorice, Uncaria rhynchophylla, Ligusticum wallichii, monkshood, Berba aristolochiae mollissimae and Caulis sinomenii have an effect of killing fungi and decomposing their hyphae, that is, they have a thrombolytic effect. Furthermore, they can treat fungi in different arteries and veins and can restore the blood circulation in the different arteries and veins.

Technical Effect

The traditional prescription the “Banxia Baizhu Tianma Decoction” has been clinically proved to be safe and effective. Using the “Banxia Baizhu Tianma Decoction” added with Acorns calamus and Curcuma longa as the basic prescription and a Chinese medicine method of adding and subtracting a decoction based on the syndrome differentiation, Sun Liting treated 14 cases of cerebral arteriosclerosis and 11 cases of vertebrobasilar insufficiency with excellent results. Using the active pharmaceutical ingredients of Gastrodia elata, Pinellia ternata, Atractylodes macrocephala, Poria cocos, orange peel and Liquorice as the basic prescription, and using a traditional method to add and subtract Uncaria rhynchophylla, Ligusticum wallichii, monkshood, Berba aristolochiae mollissimae and Caulis sinomenii according to the syndrome differentiation, the applicant treated 3 cases of apoplexy caused by deep fungal infections. After half a month of taking the medicine, the clinical symptoms of the apoplexy were significantly improved. The medicine provided by the invention is an extract of active pharmaceutical ingredients based on the traditional classical prescription “Banxia Baizhu Tianma Decoction”, wherein volatile oils and alkaloids contained in plant raw materials such as Pinellia ternata, Atractylodes macrocephala, Gastrodia elata, Poria cocos, fresh orange peel, and Liquorice are extracted to prepare a medicine or health product for treating strokes caused by fungi. Compared with traditional methods for preparing drugs, the chemical ingredients of the medicine or health product provided by the invention are known, and the effectiveness thereof is obvious.

1. A medicine for treating strokes caused by deep fungal infections, characterized in that the active pharmaceutical ingredients of the medicine comprise alkaloids and/or volatile oils extracted from Gastrodia elata, alkaloids and/or volatile oils extracted from Pinellia ternata, alkaloids and/or volatile oils extracted from Atractylodes macrocephala, alkaloids and/or volatile oils extracted from Poria cocos, alkaloids and/or volatile oils extracted from fresh orange peel, and alkaloids and/or volatile oils extracted from Liquorice; methods for preparing the medicine include: taking alkaloids of Gastrodia elata, alkaloids of Pinellia ternata, alkaloids of Atractylodes macrocephala, alkaloids of Poria cocos, alkaloids of fresh orange peel and alkaloids of Liquorice and mixing them in a certain proportion; adding various conventional excipients required to prepare different dosage forms thereto; and preparing pharmaceutically acceptable dosage forms; taking volatile oils of Gastrodia elata, volatile oils of Pinellia ternata, volatile oils of Atractylodes macrocephala, volatile oils of Poria cocos, volatile oils of fresh orange peel and volatile oils of Liquorice and mixing them in a certain proportion; adding various conventional excipients required to prepare different dosage forms thereto; and preparing pharmaceutically acceptable dosage forms; taking alkaloids and volatile oils of Gastrodia elata, alkaloids and volatile oils of Pinellia ternata, alkaloids and volatile oils of Atractylodes macrocephala, alkaloids and volatile oils of Poria cocos, alkaloids and volatile oils of fresh orange peel and alkaloids and volatile oils of Liquorice and mixing them in a certain proportion; adding various conventional excipients required to prepare different dosage forms thereto; and preparing pharmaceutically acceptable dosage forms.

2. The medicine according to claim 1, characterized in that the weight parts of the alkaloids in the active pharmaceutical ingredients of the medicine are: 0.01-50 weight parts of alkaloids of Gastrodia elata, 0.01-50 weight parts of alkaloids of Pinellia ternata, 0.01-50 weight parts of alkaloids of Atractylodes macrocephala, 0.01-50 weight parts of alkaloids of Poria cocos, 0.01-50 weight parts of alkaloids of fresh orange peel, 0.01-50 weight parts of alkaloids of Liquorice.

3. The medicine according to claim 1, characterized in that the weight parts of the volatile oils in the active pharmaceutical ingredients of the medicine are: 0.01-50 weight parts of volatile oils of Gastrodia elata, 0.01-50 weight parts of volatile oils of Pinellia ternata, 0.01-50 weight parts of volatile oils of Atractylodes macrocephala, 0.01-50 weight parts of volatile oils of Poria cocos, 0.01-50 weight parts of volatile oils of fresh orange peel, 0.01-50 weight parts of volatile oil of Liquorice.

4. The medicine according to claim 1 or 2 or 3, characterized in that a dose of the medicine to be taken daily is further preferable as: the weight of alkaloids and volatile oils contained in 50 g of Gastrodia elata, the weight of alkaloids and volatile oils contained in 35 g of Pinellia ternata, the weight of alkaloids and volatile oils contained in 30 g of Atractylodes macrocephala, the weight of alkaloids and volatile oils contained in 30 g of Poria cocos, the weight of alkaloids and volatile oils contained in 40 g of fresh orange peel, the weight of alkaloids and volatile oils contained in 25 g of Liquorice.

5. The medicine according to claim 1 or 2 or 3 or 4, characterized in that the methods for preparing the medicine include: extracting alkaloids and/or volatile oils from plant raw materials such as Gastrodia elata, Pinellia ternata, Atractylodes macrocephala, Poria cocos, fresh orange peel and Liquorice to prepare the medicine; wherein methods for extracting the alkaloids can be determined after extraction with organic solvents, and the alkaloids are extracted from plants using different solvents; supercritical fluid extraction is the first choice, followed by membrane separation, ultrasonic extraction or Soxhlet extraction; as a method for extracting the volatile oils, supercritical fluid extraction is the first choice, followed by organic solvent extraction: continuous reflux extraction or cold leaching using petroleum ether, diethyl ether or other organic solvents, or extraction using steam distillation or a cold pressing method; and preparing pharmaceutically acceptable dosage forms such as tablets, powder injections, soft capsules, granules or capsules using conventional methods for preparing pharmaceutical chemicals.

6. The medicine according to claim 1 or 2 or 4 or 5, characterized in that the methods for preparing the medicine include: according to the weight parts of the selected raw materials, extracting alkaloids from 50 g of Gastrodia elata, 35 g of Pinellia ternata, 30 g of Atractylodes macrocephala, 30 g of Poria cocos, 40 g of fresh orange peel, and 25 g of Liquorice respectively using supercritical fluid extraction or membrane separation; mixing the alkaloids and adding conventional excipients thereto; and preparing tablets according to a conventional process for preparing pharmaceutical chemicals.

7. The medicine according to claim 1 or 3 or 4 or 5, characterized in that the methods for preparing the medicine include: according to the weight parts of the selected raw materials, extracting volatile oils from 50 g of Gastrodia elata, 35 g of Pinellia ternata, 30 g of Atractylodes macrocephala, 30 g of Poria cocos, and 25 g of Liquorice respectively using supercritical fluid extraction or organic solvent extraction; extracting volatile oils from 40 g of fresh orange peel using a cold pressing method; then mixing the volatile oils and adding conventional excipients thereto; and preparing soft capsules or granules according to a conventional process for preparing pharmaceutical chemicals.

8. The medicine according to claim 1 or 2 or 3 or 4 or 5 or 6 or 7, characterized in that, according to clinical symptoms, the medicine can be selectively added with alkaloids and/or volatile oils contained in raw materials such as Uncaria rhynchophylla, Ligusticum wallichii, monkshood, Berba aristolochiae mollissimae and Caulis sinomenii.

9. The medicine according to claim 1 or 2 or 3 or 4 or 5 or 6 or 7 or 8, characterized in that the methods for preparing the medicine include: extracting alkaloids and/or volatile oils from various plant raw materials such as Gastrodia elata, Pinellia ternata, Atractylodes macrocephala, Poria cocos, fresh orange peel and Liquorice or Uncaria rhynchophylla or Ligusticum wallichii or monkshood or Berba aristolochiae mollissimae or Caulis sinomenii to prepare the medicine; and preparing pharmaceutically acceptable dosage forms such as tablets, powder injections, soft capsules, granules or capsules using conventional methods for preparing pharmaceutical chemicals.

10. The medicine according to claim 1 or 2 or 3 or 4 or 5 or 6 or 7 or 8 or 9, characterized in that the scope of application of the medicine includes applications in medical drugs, health foods, health care products, animal drugs, and animal feed.
